(19) United States
(12) Patent Application Publication
(10) Pub. No.: US 2021/0302453 A1
(43) Pub. Date: Sep. 30, 2021

(54) MEDICAL INFORMATION PROCESSING APPARATUS AND MEDICAL INFORMATION MANAGEMENT METHOD

(71) Applicant: OLYMPUS CORPORATION, Tokyo (JP)

(72) Inventor: Tomoaki KITAZAKI, Tokyo (JP)

(73) Assignee: OLYMPUS CORPORATION, Tokyo (JP)

(21) Appl. No.: 17/346,592

(22) Filed: Jun. 14, 2021

Related U.S. Application Data
(63) Continuation of application No. PCT/JP2018/045937, filed on Dec. 13, 2018.

(51) Int. Cl. G01N 35/02; G16H 10/40

(52) U.S. Cl. CPC G01N 35/02; G01N 2035/00782; G16H 10/40

(57) ABSTRACT

An acquirer acquires information regarding an examination. An association unit associates the information regarding the examination with identification information of a sample container. A status management unit sets the status of the sample container based on information regarding the movement of the sample container. The status of the sample container may include a reserved-for-use status in which the sample container is reserved for use in an associated examination and a used status in which the sample container has been used in the associated examination. When the association unit associates the information regarding the examination with the identification information of the sample container, the status management unit sets the status of the sample container to the reserved-for-use status.

### CROSS-REFERENCE TO RELATED APPLICATIONS

This application is based upon and claims the benefit of priority from International Application No. PCT/JP2018/045937, filed on Dec. 13, 2018, the entire contents of which are incorporated herein by reference.

### BACKGROUND

### 1. Technical Field

The present disclosure relates to a technology for managing a status of a sample container.

### 2. Description of the Related Art

Japanese Patent Application Publication No. 2005-9863 discloses a sample rack provided with a plurality of housing units for housing a plurality of sample containers. The sample containers have an IC tag for storing sample information, and an antenna for receiving the sample information stored in the IC tag is arranged at the bottom of the housings. Each antenna is connected to an IC tag R/W, and sample information is received by the antenna and read by the IC tag R/W.

In a flow in which a pathologist makes a definitive diagnosis on a biological tissue, a medical examiner first collects the patient's tissue with an endoscope or the like, and the collected tissue is placed in a sample container.

After the examination is completed, the sample container is taken to a pathology room, and a pathologist cuts out a part suitable for diagnosis from the tissue to prepare a tissue specimen. The pathologist observes the tissue specimen under a microscope to make a pathological diagnosis.

### SUMMARY

Pathological sample handling manuals are prepared at each medical facility in order to prevent mixing up of samples. The manuals stipulate that regarding a step of putting a tissue into a sample container, a label on which patient information such as a patient name and a patient ID is written is attached to the container main body before putting the tissue into the sample container. Attaching a label later and attaching a label on a lid are prohibited for the purpose of preventing mixing up, and a medical staff thoroughly follows a procedure of putting a collected tissue in a sample container after confirming the patient information on the attached label.

However, since the printing and attaching of labels is done manually by a medical staff, human errors such as losing labels and mislabeling may occur. Therefore, apart from following the handling manuals, it is required to build a mechanism for reducing the possibility of mixing up of samples.

One embodiment of the present disclosure relates to a medical information processing apparatus for managing a status of a sample container having identification information, and the medical information processing apparatus includes: an acquirer that acquires information regarding an examination; an association unit that associates the information regarding the examination with the identification information of the sample container; and a status management unit that sets the status of the sample container. The status management unit sets the status of the sample container based on information regarding the movement of the sample container.

Optional combinations of the aforementioned constituting elements and implementations of the present disclosure in the form of methods, apparatuses, systems, recording mediums, and computer programs may also be practiced as additional modes of the present disclosure.

### BRIEF DESCRIPTION OF THE DRAWINGS

Embodiments will now be described, by way of example only, with reference to the accompanying drawings that are meant to be exemplary, not limiting, and wherein like elements are numbered alike in several figures, in which:

FIG. 1 is a diagram showing the configuration of a medical support system according to an embodiment;

FIG. 2 is a diagram showing functional blocks of a medical information processing apparatus;

FIG. 3 is a diagram showing a schematic cross section of a sample container and a tray; and

FIG. 4 is a diagram showing functional blocks of a processing apparatus.

### DETAILED DESCRIPTION

The disclosure will now be described by reference to the preferred embodiments. This does not intend to limit the scope of the present disclosure, but to exemplify the disclosure.

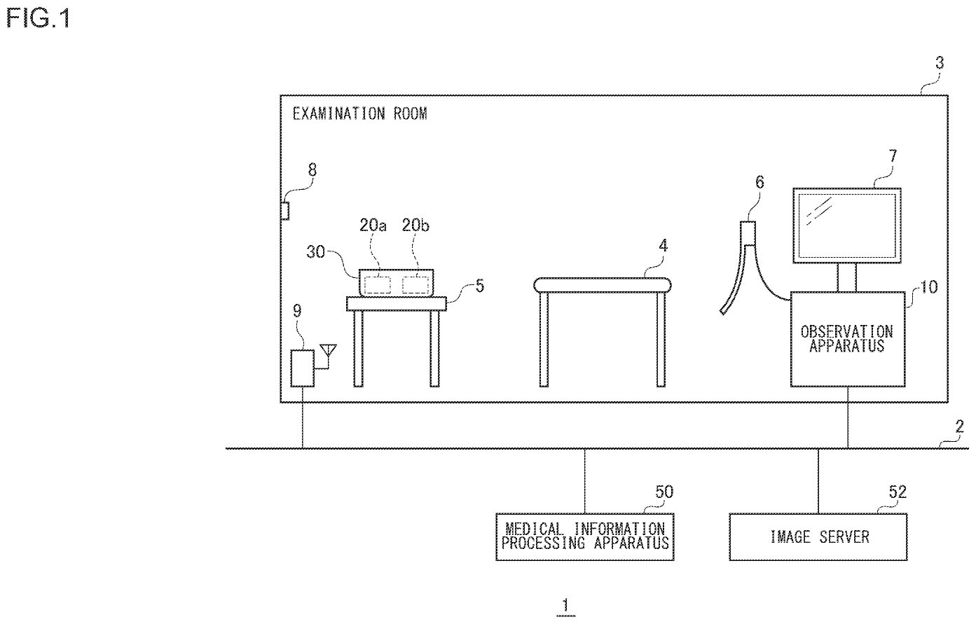

FIG. 1 shows the configuration of a medical support system 1 according to an embodiment. The medical support system 1 includes an observation apparatus 10, a medical information processing apparatus 50, and an image server 52, which are interconnected by a network 2 such as a local area network (LAN). The medical information processing apparatus 50 may be a management server that manages information regarding examinations.

An examination room 3 is provided with a patient bed 4, a placing table 5 for placing objects, an examination room radio frequency identifier (RFID) tag 8, an access point (AP) 9, and an observation apparatus 10. Before the start of an endoscopic examination, medical staff member places one or more sample containers 20a and 20b (hereinafter referred to as “sample containers 20” unless otherwise distinguished) on a tray 30, brings the sample containers 20 on the tray to the examination room 3, and places the sample containers 20 on the tray 30 on the placing table 5. The placing table 5 may be used as a working table when the tissue is put in the sample container 20 and is preferably arranged at a position close to the patient bed 4.

Although FIG. 1 shows only one examination room 3, the medical facility may have a plurality of examination rooms. The AP9 has a wireless LAN function and can communicate with a wireless LAN communication device. The examination room RFID tag 8 records identification information (examination room ID) for identifying the examination room, and when the RFID reader is brought close to the examination room RFID tag 8, the examination room ID is read out by the RFID reader. The examination room RFID tag 8 may be installed near the entrance of the examination room 3. As will be described later, the tray 30 is provided with an RFID reader and a wireless LAN communication module, and a medical staff member who brings the tray 30 to the examination room 3 causes the RFID reader to read the examination room ID of an examination room RFID tag 8 when entering the room and causes the examination room ID to be transmitted from the wireless LAN communication module to the medical information processing apparatus 50. This allows the medical information processing apparatus 50 to realize that the tray 30 has entered the examination room 3.

An endoscope 6 is connected to the observation apparatus 10. The endoscope 6 is provided with a solid-state imaging device (for example, CCD image sensor or CMOS image sensor) and a signal processing circuit. The solid-state imaging device converts incident light into an electrical signal, and the signal processing circuit performs signal processing such as A/D conversion, noise removal, and the like on image data photoelectric-converted by the solid-state imaging device and outputs the resulting image data to the observation apparatus 10. The endoscope 6 captures a still image of the inside of a body at the time when the medical examiner presses a release switch.

One important role of the observation apparatus 10 is to transmit an examination image captured by the endoscope 6 to the image server 52 so as to store the examination image. Another important role is to display video acquired by the endoscope 6 on a display 7 in real time. In the former role, when the release switch of the endoscope 6 is pressed, the observation apparatus 10 adds identification information for identifying the examination (examination ID) and image-capturing time information indicating the image-capturing time to an image captured by the endoscope 6 at least as metadata so as to generate examination image data. The observation apparatus 10 transmits the examination image data to the image server 52, and the image server 52 records captured-image data.

The medical information processing apparatus 50 according to the embodiment has a function of managing the status of the sample container 20. The sample container 20 is a petri dish or a test tube into which a tissue (sample) collected during an endoscopic examination or the like is put, and the sample container 20 is put on the tray 30 and brought into the examination room 3. One sample is basically put in the sample container 20. However, regarding samples collected from the same site, a plurality of samples may be put in the sample container 20.

In the embodiment, the medical information processing apparatus 50 manages the sample container 20 according to the following four statuses.

(1) Unreserved

An “unreserved” status indicates a state where no examination is assigned to the sample container 20, and the sample container 20 is in a state of not being scheduled to be used. The “unreserved” status indicates a state where the sample container 20 is in a state of being able to be assigned to an examination. In a state where the sample container 20 is stored in a container shelf or in a state where the sample container 20 is brought into the examination room 3 before the start of the examination, the status of the sample container 20 is indicated as the “unreserved” status.

(2) Reserved for Use

A “reserved for use” status indicates a state where an examination has been assigned to the sample container 20, and the sample container 20 has been reserved for use in the examination. The “reserved for use” status indicates a state where the sample container 20 cannot be assigned to another examination. When an examination is assigned to the sample container 20, the status of the sample container 20 changes from the “unreserved” status to the “reserved for use” status. In the “reserved for use” status, it is not confirmed whether or not the sample container 20 will be actually used. Therefore, if the sample container 20 is not used for an examination and the assignment to the examination is cancelled, the status of the sample container 20 returns from the “reserved for use” status to the “unreserved” status.

(3) Possibility of being Used

A “possibility of being used” status indicates a state where a possibility of the sample container 20 being used during an examination has arisen. As will be described later, the arising of the possibility of the sample container 20 being used is determined based on information regarding the movement of the sample container 20 with respect to the tray 30. As an example, when the sample container 20 that is placed on the tray 30 is taken out from the tray 30, the status of the sample container 20 changes from the “reserved for use” status to the “possibility of being used” status.

(4) Used

A “used” status indicates a state of having been used for an examination. The state of having been used for an examination is determined based on the fact that the sample container 20 in the “possibility of being used” status has been returned to the tray 30. After the completion of the examination, the sample container 20 in the “used” status is taken to a pathology room and used to prepare a tissue specimen.

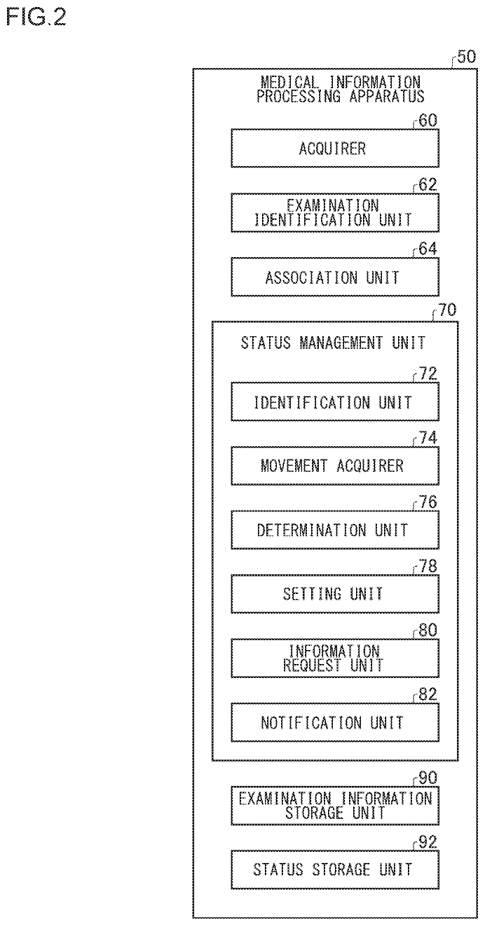

FIG. 2 shows functional blocks of the medical information processing apparatus 50. The medical information processing apparatus 50 includes an acquirer 60, an examination identification unit 62, an association unit 64, a status management unit 70, an examination information storage unit 90, and a status storage unit 92. The status management unit 70 has a function of setting the status of the sample container 20 based on the information regarding the movement of the sample container 20 and has an identification unit 72, a movement acquirer 74, a determination unit 76, a setting unit 78, an information request unit 80, and a notification unit 82. The examination information storage unit 90 stores examination information, and the status storage unit 92 stores the latest status of the sample container 20.

The configuration thereof is implemented by hardware such as an arbitrary processor, a memory, or other LSIs and by software such as a program or the like loaded into the memory. The figure depicts functional blocks implemented by the cooperation of hardware and software. Thus, a person skilled in the art should appreciate that there are many ways of accomplishing these functional blocks in various forms in accordance with the components of hardware only, software only, or the combination of both.

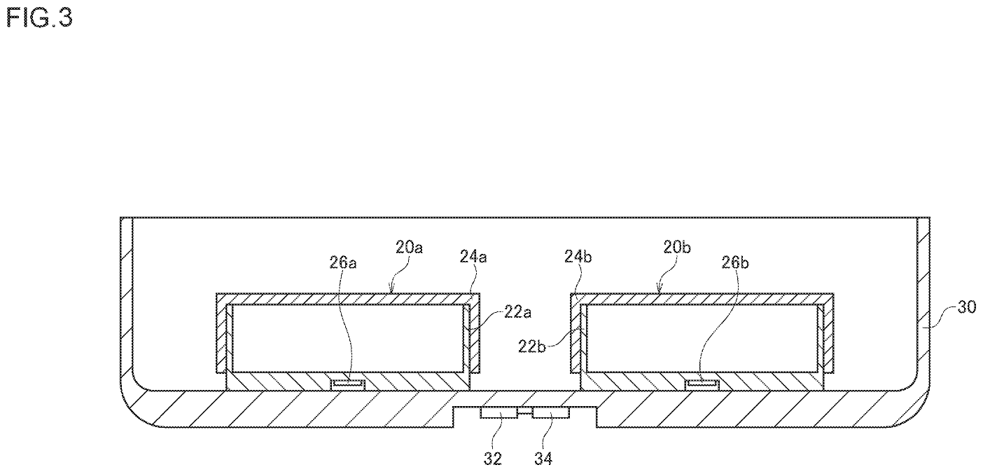

FIG. 3 shows a schematic cross section of the sample container 20 and the tray 30. In this figure, two sample containers 20a and 20b are placed on the tray 30. The sample container 20a has a container main body 22a and a lid 24a. An RFID tag 26a is an IC tag in which identification information (container ID: petri_dish_a) of the sample container 20a is recorded and is attached to a recessed region on the bottom surface of the container main body 22a. The RFID tag 26a may be attached to the side surface of the container main body. The RFID tag 26a is a passive type IC tag and may operate using radio waves from an RFID reader as an energy source.

In the same manner, the sample container 20b has a container main body 22b and a lid 24b. An RFID tag 26b is an IC tag in which identification information (container ID: petri_dish_b) of the sample container 20b is recorded and is attached to a recessed region on the bottom surface of the container main body 22b. The RFID tag 26b may be a passive type IC tag. Hereinafter, when not particularly distinguished, the container main bodies 22a and 22b are sometimes referred to as “container main bodies 22”, the lids 24a and 24b are sometimes referred to as “lids 24”, and the RFID tags 26a and 26b are sometimes referred to as “RFID tags 26”.

The tray 30 is a storage container having a relatively shallow bottom with an open top surface, and an RFID reader 32 and a processing apparatus 34 are attached to the bottom surface. The RFID reader 32 and the processing apparatus 34 are supplied with electric power from a power source (not shown). The RFID reader 32 emits radio waves to the surroundings and receives reflected waves from the RFID tags 26a and 26b. The reflected wave from the RFID tag 26a includes the container ID (petri_dish_a), and the reflected wave from the RFID tag 26b includes the container ID (petri_dish_b). The RFID reader 32 reads the container IDs from the received reflected waves and supplies the container IDs to the processing apparatus 34 along with the reception intensity at that time. The tray 30 is an example of a container for storing the sample container 20, and the RFID reader 32 and the processing apparatus 34 may be provided in a storage container of a different type.

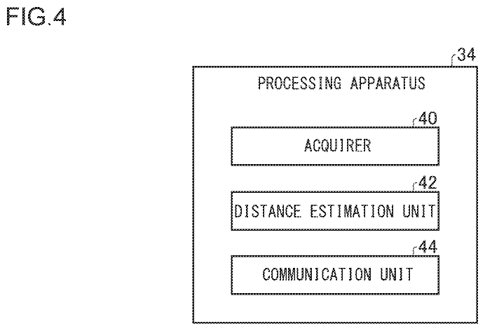

FIG. 4 shows functional blocks of the processing apparatus 34. The processing apparatus 34 includes an acquirer 40, a distance estimation unit 42, and a communication unit 44. The communication unit 44 is a wireless LAN communication module and connects to the AP9. The acquirer 40 acquires a container ID and reception intensity from the RFID reader 32. The distance estimation unit 42 estimates the distance between a container main body 22 identified by the container ID and the RFID reader 32 using the reception intensity and passes estimated distance information to the communication unit 44. The distance estimation unit 42 may estimate the distance between the container main body 22 and the RFID reader 32 when the reception intensity of a reflected wave from the same container main body 22 changes by a predetermined value or a predetermined ratio or more. As a result of this, if the change in reception intensity is small, the distance estimation unit 42 does not have to perform a distance estimation process, and power consumption can therefore be suppressed.

A distance estimation function of the distance estimation unit 42 may be provided in the RFID reader 32, and the RFID reader 32 may calculate the estimated distance. In this case, the RFID reader 32 supplies the processing apparatus 34 with the container ID of the RFID tag 26 and the estimated distance information.

Before the start of the examination, the medical staff member takes to the examination room 3 the tray 30 on which the sample containers 20a and 20b are placed. At the time of entering the examination room 3, the medical staff member brings the tray 30 close to the examination room RFID tag 8. The examination room RFID tag 8 is a passive type IC tag. Upon receiving a radio wave from the RFID reader 32, the examination room RFID tag 8 transmits the identification information (examination room ID) of the examination room 3 to the RFID reader 32. The examination room RFID tag 8 may be an active type IC tag. The examination room RFID tag 8 may be of any type and transmits the examination room ID to the RFID reader 32.

The RFID reader 32 supplies the received examination room ID of the examination room 3 to the processing apparatus 34. When the acquirer 40 acquires the examination room ID, the communication unit 44 transmits the examination room ID to the medical information processing apparatus 50 along with the identification information (tray ID) of the tray 30.

Referring back to FIG. 2, in the medical information processing apparatus 50, the acquirer 60 acquires the tray ID of the tray 30 and the examination room ID of the examination room 3 and supplies the tray ID and the examination room ID to the status management unit 70. The identification unit 72 finds out that the tray 30 has been brought into the examination room 3 based on the supplied tray ID and examination room ID.

When it is found out that the tray 30 has been brought into the examination room 3, the information request unit 80 requests the tray 30 to transmit the identification information of the sample container 20 stored on the tray 30. When the acquirer 40 acquires the transmission request in the processing apparatus 34, the communication unit 44 transmits the container IDs of the sample containers 20a and 20b supplied from the RFID reader 32 to the medical information processing apparatus 50. In the medical information processing apparatus 50, the acquirer 60 acquires the tray ID of the tray 30, the container ID (petri_dish_a) of the sample container 20a, and the container ID (petri_dish_b) of the sample container 20b. The identification unit 72 finds out that the two sample containers 20a and 20b are stored on the tray 30.

At this time, the association unit 64 determines whether or not an examination is being performed in the examination room 3. If the examination is being performed, the association unit 64 associates the sample containers 20a and 20b brought into the examination room 3 with the examination being performed in the examination room 3. In this example, the identification unit 72 identifies the container IDs of the two sample containers 20a and 20b before the start of the examination, and the examination has not yet started at this point. The medical staff member brings the tray 30 on which the sample containers 20a and 20b are placed close to the examination room RFID tag 8 and then places the tray 30 on the placing table 5.

Before the start of the examination, the medical staff member specifies an examination order for an examination to be performed. The observation apparatus 10 displays a list of examination orders for the day on the display 7, and the medical staff member selects one examination order from the list. Alternatively, another terminal device may be installed in the examination room 3, and the examination order may be selected through the terminal device. The examination order includes information regarding the examination such as examination identification information (examination ID), a scheduled examination date, an examination type, patient information, a medical examiner, and a scheduled examination time. For example, a patient may wear a wristband on which patient information is recorded on his/her arm, and the patient information of the wristband may be read by a reader device so as to automatically select an examination order issued to the patient.

The information of the selected examination order is transmitted to the medical information processing apparatus 50 along with the examination room ID. The acquirer 60 acquires the examination room ID and the information of the examination order and stores the examination room ID and the information on the examination order in the examination information storage unit 90. As described above, the information of the examination order includes information on the examination such as an examination ID, a scheduled examination date, an examination type, patient information, a medical examiner, and a scheduled examination time.

In the examination room 3, when the endoscope 6 is connected to the observation apparatus 10 and an examination start button of the observation apparatus 10 is operated, the observation apparatus 10 transmits information regarding the start of the examination (hereinafter, also referred to as “examination start information”) to the medical information processing apparatus 50. When the acquirer 60 acquires the examination start information, the acquirer 60 stores the examination start time in the examination information storage unit 90 in association with the examination ID. At the time of the completion of the examination, when information regarding the completion of the examination (hereinafter, also referred to as “examination completion information”) is transmitted from the observation apparatus 10, the acquirer 60 stores the examination completion time in the examination information storage unit 90 in association with the examination ID.

When the acquirer 60 acquires the examination start information, the association unit 64 associates the information regarding the examination with the container ID of the sample container 20 based on the examination room ID. In this example, the association unit 64 associates the container IDs of the sample containers 20a and 20b with information regarding the examination performed in the examination room 3. The information regarding the examination associated in this case includes the examination ID and may further include the patient information. The container ID and the associated information regarding the examination may be stored in the status storage unit 92.

When the association unit 64 associates the information regarding the examination with the container ID of the sample container 20, the setting unit 78 sets the status of the sample container 20 to the “reserved for use” status. The “reserved for use” status indicates a state where an examination has been assigned to the sample container 20. The status storage unit 92 stores the latest status of all the sample containers 20 in the medical facility, and the setting unit 78 updates a status value in the status storage unit 92 when the status of the sample containers 20 is changed.

The information request unit 80 requests the tray 30 to transmit estimated distance information of the sample containers 20. When the acquirer 40 acquires the transmission request in the processing apparatus 34, the communication unit 44 transmits the estimated distance information of each of the sample containers 20a and 20b calculated by the distance estimation unit 42 to the medical information processing apparatus 50. In the medical information processing apparatus 50, the acquirer 60 acquires the tray ID of the tray 30, the estimated distance information of the sample container 20a, and the estimated distance information of the sample container 20b and transmits the tray ID and the pieces of estimated distance information to the movement acquirer 74.

The movement acquirer 74 acquires the estimated distance information of the sample containers 20 as information regarding the movement of the sample containers 20 with respect to the RFID reader 32 of the tray 30. In the embodiment, the distance estimation unit 42 in the processing apparatus 34 calculates the estimated distance. Alternatively, the movement acquirer 74 may acquire the reception intensity in the RFID reader 32 from the processing apparatus 34 and estimate the distance between container main body 22 and the RFID reader 32 for each sample container 20.

The movement acquirer 74 may periodically acquire the estimated distance information. In this case, the information request unit 80 may periodically request the tray 30 to transmit the estimated distance information of the sample containers 20, and the processing apparatus 34 may be programmed to periodically transmit the estimated distance information. When the distance estimation unit 42 calculates the estimated distance when the reception intensity changes by a predetermined value or a predetermined ratio or more, the communication unit 44 may transmit the estimated distance information each time the distance estimation unit 42 calculates the estimated distance to the medical information processing apparatus 50, and the movement acquirer 74 may acquire the estimated distance information.

Using the estimated distance information, the determination unit 76 determines whether or not a sample container 20 has been taken out from the tray 30. The removal of the sample container 20 from the tray 30 means that a possibility of the sample container 20 going to be used as a container for a collected tissue has arisen. The determination unit 76 may determine whether or not the sample container 20 has been separated from the tray 30 by using the distance indicated by the estimated distance information or may determine whether or not the sample container 20 has been separated from the tray 30 based on the amount of change in the estimated distance acquired in chronological order.

With reference to FIG. 3, when the sample container 20 is placed on the tray 30, the maximum distance between the RFID tag 26 and the RFID reader 32 is about 30 cm in the case of a normal tray 30 size. When an absolute distance is used as the determination criterion, the determination unit 76 may determine that the sample container 20 has been taken out from the tray 30 when the estimated distance is, for example, 50 cm or more.

Further, while the sample container 20 is placed on the tray 30, the distance between the RFID tag 26 and the RFID reader 32 does not change basically. Therefore, the determination unit 76 may determine that the sample container 20 has been taken out from the tray 30 when the amount of change from the distance at that time exceeds a predetermined value.

When the determination unit 76 determines that the sample container 20 has been taken out from the tray 30, the setting unit 78 sets the status of the sample container 20 to the “possibility of being used” status and updates the status value in the status storage unit 92. The “possibility of being used” status indicates a state where a possibility of the sample container 20 being used has arisen.

An ID readable range of the RFID reader 32 is adjusted according to the intensity of the radio wave transmitted from the RFID reader 32. Therefore, a level of transmission intensity may be set that prevents a reflected wave from the RFID tag 26 from reaching the RFID reader 32 when the sample container 20 is taken out from the tray 30. When the RFID reader 32 can no longer receive a container ID, the processing apparatus 34 transmits the container ID that can no longer be received to the medical information processing apparatus 50, and the movement acquirer 74 acquires information indicating that the container ID can no longer be received as information regarding the movement of the container 20. When the container ID can no longer be received, the determination unit 76 may determine that the sample container 20 has been taken out from the tray 30.

The determination unit 76 determines whether or not the sample container 20 in the “possibility of being used” status has been returned to the tray 30. In the same way as in the criterion used when determining whether the sample container 20 has been separated from the tray 30, the determination unit 76 may determine whether the sample container 20 has been returned to the tray 30 based on the distance indicated by the estimated distance information or on the amount of change in the estimated distance acquired in chronological order. For example, the determination unit 76 may store the minimum value and the maximum value of the estimated distance before the sample container 20 becomes separated from the tray 30, and when the estimated distance is within the range between the minimum value and the maximum value, the determination unit 76 may determine that the sample container 20 has been returned to the tray 30.

When a sample container 20 having a container ID different from those of the sample containers 20a and 20b is placed on the tray 30, the identification unit 72 finds out that a sample container 20 not subject to management has been placed on the tray 30. The sample container 20 not subject to management is a sample container that is not associated with an examination performed in the examination room 3 and includes, for example, a container associated with an examination performed in another examination room. At this time, the notification unit 82 preferably give notification indicating that a sample container 20 not subject to management has been placed to a portable terminal device or the like possessed by the medical staff member.

When the determination unit 76 determines that the sample container 20 has been returned to the tray 30, the setting unit 78 sets the status of the sample container 20 to the “used” status and updates the status value in the status storage unit 92. The “used” status indicates a state of where the sample container 20 has been used for an examination.

When examination completion information is transmitted from the observation apparatus 10 at the completion of the examination, the setting unit 78 checks the final status of the sample containers 20a and 20b associated with the examination. At this time, if the final status is “reserved for use” or “used”, there is no problem. However, if the final status is “possibility of being used”, the sample container 20 has not been returned to the tray 30. Therefore, the notification unit 82 preferably give notification to the portable terminal device or the like possessed by the medical staff member indicating that the sample container 20 has not been returned to the tray 30.

If the status at the time of the completion of the examination is the “reserved for use” status, the setting unit 78 changes the “reserved for use” status to the “unreserved” status. As a result, the assignment to the examination is cancelled, and the sample container 20 becomes available to be assigned to another examination.

By storing the latest status of a sample container 20 in the status storage unit 92 as described above, mixing up of samples can be prevented. For example, when a sample container 20 is taken to a pathology room and a pathologist reads the container ID of the sample container 20 with an RFID reader, information regarding an examination associated with the sample container 20 may be displayed on a terminal device in the pathology room. Thereby, even if a wrong label is attached to a sample container 20, the pathologist notices that a human error has occurred if the status thereof is different from the status of the sample container 20 that is managed.

Described above is an explanation on the present disclosure based on the embodiments. These embodiments are intended to be illustrative only, and it will be obvious to those skilled in the art that various modifications to constituting elements and processes could be developed and that such modifications are also within the scope of the present disclosure.

In the embodiments, an RFID tag 26 is attached to a container main body 22. However, an RFID tag may also be attached to a lid 24. The distance estimation unit 42 calculates the estimated distance between the container main body 22 and the RFID reader 32 and the estimated distance between the lid 24 and the RFID reader 32, and the communication unit 44 transmits the respective pieces of estimated distance information to the medical information processing apparatus 50.

The determination unit 76 determines whether or not the lid 24 has been removed from the container main body 22 based on the relationship between the estimated distance of the container main body 22 and the estimated distance of the lid 24. When a tissue is put into the container main body 22, the lid 24 is always removed from the container main body 22. Therefore, on condition that the difference between the estimated distance of the lid 24 and the estimated distance of the container main body 22 exceeds a predetermined value based on the two pieces of estimated distance information, the setting unit 78 may set the status of the sample container 20 to the “used” status when the sample container 20 is returned to the tray 30.

Further, in the embodiments, the RFID reader 32 is attached to the tray 30, which is the storage container of the sample container 20. However, an RFID reader may be attached to the placing table 5 of the storage container.

What is claimed is:

1. A medical information processing apparatus for managing a status of a sample container having identification information, comprising: a processor comprising hardware, wherein
the processor is configured to:
acquire information regarding an examination to be performed;
associate the information regarding the examination with the identification information of the sample container;
set the status of the sample container to a reserved-for-use status that indicates that the sample container cannot be assigned to another examination;
upon acquiring information indicating that the sample container has been moved when the status is set to the reserved-for-use status, change the status from the reserved-for-use status to a possibility-of-being-used status; and
upon acquiring information regarding the completion of the examination when the status is set to the reserved-for-use status, change the status from the reserved-for-use status to an unreserved status that indicates that the sample container can be assigned to another examination.

2. The medical information processing apparatus according to claim 1, wherein
the status of the sample container includes a used status in which the sample container has been used in an associated examination.

3. The medical information processing apparatus according to claim 2, wherein
the processor is configured to:
set the status of the sample container to the reserved-for-use status when the information regarding the examination becomes associated with the identification information of the sample container.

4. The medical information processing apparatus according to claim 3, wherein
the processor is configured to:
set the status of the sample container according to the distance between the sample container and a reader for the identification information.

5. The medical information processing apparatus according to claim 4, wherein
the processor is configured to:
change the reserved-for-use status to another status when the distance between the sample container and the reader for the identification information changes.

6. The medical information processing apparatus according to claim 1, wherein
the processor is configured to:
notify a user upon acquiring the information regarding the completion of the examination when the status is set to the possibility-of-being-used status.

7. The medical information processing apparatus according to claim 5, wherein
the processor is configured to:
change the status of the sample container from the reserved-for-use status to the possibility-of-being-used status when a tray for placing the sample container and the sample container become separated from each other; and
when the tray and the sample container become close to each other after the status is changed to the possibility-of-being-used status, change the status of the sample container from the possibility-of-being-used status to the used status.

8. The medical information processing apparatus according to claim 7, wherein
the processor is configured to:
notify a user upon acquiring the information regarding the completion of the examination when the status is set to the possibility-of-being-used status; and
not notify the user upon acquiring the information regarding the completion of the examination when the status is set to the used status.

9. The medical information processing apparatus according to claim 5, wherein
the processor is configured to:
change the status of the sample container from the reserved-for-use status to the possibility-of-being-used status when a tray for placing the sample container and the sample container become separated from each other; and
notify a user when the tray and a sample container not associated with the examination become close to each other after the status is changed to the possibility-of-being-used status.

10. The medical information processing apparatus according to claim 1, wherein
the processor is configured to:
associate the information regarding the examination with the identification information of the sample container when the status is set to the unreserved status.

11. A medical information management method for managing a status of a sample container having identification information, comprising:
acquiring information regarding an examination to be performed;
associating the information regarding the examination with the identification information of the sample container;
setting the status of the sample container to a reserved-for-use status that indicates that the sample container cannot be assigned to another examination;
upon acquiring information indicating that the sample container has been moved when the status is set to the reserved-for-use status, changing the status from the reserved-for-use status to a possibility-of-being-used status; and
upon acquiring information regarding the completion of the examination when the status is set to the reserved-for-use status, changing the status from the reserved-for-use status to an unreserved status that indicates that the sample container can be assigned to another examination.

12. The medical information management method according to claim 11, wherein
the status of the sample container includes a used status in which the sample container has been used in an associated examination.

13. The medical information management method according to claim 12, comprising:
setting the status of the sample container to the reserved-for-use status when the information regarding the examination becomes associated with the identification information of the sample container.

14. The medical information management method according to claim 13, comprising:
setting the status of the sample container according to the distance between the sample container and a reader for the identification information.

15. The medical information management method according to claim 14, comprising:
changing the reserved-for-use status to another status when the distance between the sample container and the reader for the identification information changes.

16. The medical information management method according to claim 11, comprising:
notifying a user upon acquiring the information regarding the completion of the examination when the status is set to the possibility-of-being-used status.

17. The medical information management method according to claim 15, comprising:
changing the status of the sample container from the reserved-for-use status to the possibility-of-being-used status when a tray for placing the sample container and the sample container become separated from each other; and
when the tray and the sample container become close to each other after the status is changed to the possibility-of-being-used status, changing the status of the sample container from the possibility-of-being-used status to the used status.

18. The medical information management method according to claim 17, comprising:
notifying a user upon acquiring the information regarding the completion of the examination when the status is set to the possibility-of-being-used status; and
not notifying the user upon acquiring the information regarding the completion of the examination when the status is set to the used status.

19. The medical information management method according to claim 15, comprising:
changing the status of the sample container from the reserved-for-use status to the possibility-of-being-used status when a tray for placing the sample container and the sample container become separated from each other; and
notifying a user when the tray and a sample container not associated with the examination become close to each other after the status is changed to the possibility-of-being-used status.

20. The medical information management method according to claim 11, comprising:
associating the information regarding the examination with the identification information of the sample container when the status is set to the unreserved status.
